(19) United States
(12) Patent Application Publication
(10) Pub. No.: US 2008/0044838 A2
(43) Pub. Date: Feb. 21, 2008

(54) Molecular Spacer Arm, Process for the Production Thereof and Uses on an Analytical Chip Comprising Molecules or Biomolecules

(75) Inventors: Ve´ronique PERETTI, Grenoble (FR); Françoise VINET, Grenoble (FR); David BONNAFFE, Paris (FR)

Correspondence Address: HUTCHISON LAW GROUP PLLC, PO BOX 31686, RALEIGH, NC, 27612, US

(73) Assignee: Commissariat A L'Energie Atomique, Paris (FR)

(21) Appl. No.: 10/590,137

(22) Filed: Aug. 22, 2006

(30) Foreign Application Priority Data
Feb. 25, 2004 (FR) 04 50346

(51) Int. Cl. G01N 33/53; C08B 37/00; C07K 14/775; C07K 14/47

(52) U.S. Cl. 435/7.1; 435/287.2; 536/53; 530/359; 530/395

(57) ABSTRACT

The present invention relates to a molecular spacer arm, to a process for attachment of a molecular unit to a solid support, and also to the use of this spacer arm on analytical chips comprising molecules or biomolecules. The spacer arm has the formula (I): 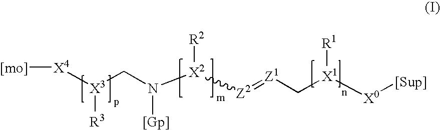
in which X⁰, X⁴═C, O, S, Se, N, P, As; X¹⁻³═C, O, N, S, Se, P, As, or C₁₋₆ aryl or heteroaryl; Z¹⁻²═C—R, Si—R, N, P and As, where R═C₁₋₆ alkyl; R¹⁻³═H, or C₁₋₆ alkyl, aryl or heteroaryl; [Gp]=protective group for >N; n, m and p=integers ≧1; [Sup]=H or a silanized solid support; and [mo]=H or a molecular unit intended to be covalently attached to said silanized solid support by means of said spacer arm.

### TECHNICAL FIELD

The present invention relates to a molecular spacer arm, to a process for preparing the spacer arm connecting a molecular unit to a solid support, and to the use of this spacer arm on analytical chips comprising molecules or biomolecules.

In the disclosure which follows, references between square brackets [ ] refer to the reference list at the end of the description.

The analytical chips targeted by the present invention are more particularly, but not exclusively, biochips and Microsystems dedicated to biological analysis. They can be divided into three categories: DNA chips, lab-on-chips and cell-on-chips.

Currently, a new type of biochip is emerging: the glycochip. This biochip is either the result of a deposition of a natural or synthetic substance, or the result of a supported multiparallel synthesis (combinatorial chemistry) of various oligosaccharide sequences, representative of the molecular diversity of certain large families of endogenous glycoconjugates, for example heparan sulphates. The present invention is particularly suitable for this new type of biochip because it allows in particular the attachment of these molecules to biochip supports by means of a chemical process which is effective and simplified compared with the prior art.

The molecules or biomolecules, hereby called “molecular units”, which can be attached to a solid support by means of the spacer arm of the present invention may, for example, be nucleic acids (DNA or RNA), sugars, glycoproteins, glycolipids, etc. Other further examples are given below.

### PRIOR ART

In the majority of biochips, a spacer provides the link between the solid support [2] and, for example, the oligopeptide, oligonucleotide [3] or oligosaccharide [4] probes. This spacer can play several roles at a time: linking molecule, spatial distancing arm, site of cleavage of the probe, etc.

The proximity of the support to the sites of recognition of the targets by the probes can in fact hinder, or even prevent, the probe/target recognition, and therefore harm the fineness and the quality of analysis of the biochips. This is particularly true when the probes are small, for example in the case of glycochips.

The equation of principle below indicates the general scheme for the formation and then cleavage of the spacer, in which X′ represents a solid support, and X″ a molecular unit.

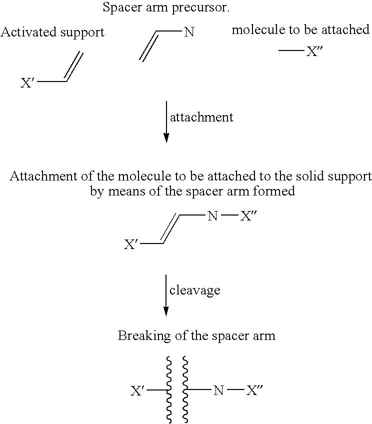

Numerous spacer arms have been produced to date, but they have a certain number of unresolved drawbacks. Specifically, their structure implies a severe limitation on the chemical processes which can be used for attaching them to the solid support and/or they do not make it possible to readily attach any type of biological molecule and/or they are so chemically stable that, once attached to the solid support, they cannot be readily cleaved in order to recover the biological molecule, and said cleavage can lead to the deterioration of said molecule or of the support.

Document [4] (U.S. Pat. No. 6,579,725) describes a spacer arm for attaching oligosaccharides. This spacer arm, although it is more effective than those of the even more prior art, does not make it possible to solve all the abovementioned problems at once. It may also be noted that the length, functionality, reactivity and hindrance thereof cannot always be generated as desired.

### DISCLOSURE OF THE INVENTION

The present invention makes it possible, precisely, to solve the abovementioned problems of the prior art all at once, by providing a molecular spacer arm of formula (I) below:

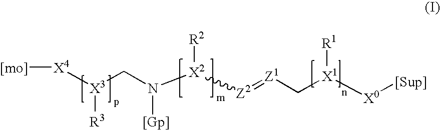

in which the substituents X⁰; X¹; X²; X³; X⁴; Z¹; Z²; R¹; R²; and R³ are such that:
X⁰ and X⁴ are each chosen, independently of the other substituents, from C, O, N, S, Se, P, As and Si;
X¹; X²; and X³ are each chosen, independently of the other substituents, from C, O, N, S, Se, P, As and Si, and from an aryl and a heteroaryl, each containing, for example, from 2 to 20 carbon atoms;
Z¹ and Z² are each chosen, independently of the other substituents, from C—R, Si—R, C, N, P and As, where R is an alkyl containing, for example, from 1 to 40 carbon atoms;

R¹; R²; and R³ are each chosen, independently of the other substituents, from H, an alkyl, an aryl and a heteroaryl each containing from 2 to 20 carbon atoms;
[Gp] represents a group which protects the secondary amine —N— or a molecule which participates in the functionality of the spacer arm;

in which n, m and p are integers, each greater than or equal to 1 and chosen independently of one another, preferably such that 1≦n, m and p≦40;

in which [Sup] represents H or a silanized solid support to which said spacer arm can be covalently attached; and

in which [mo] represents H or a molecular unit intended to be covalently attached by means of said spacer arm to said silanized solid support.

Of course, X⁰ to X⁴ are atoms which form the backbone of the spacer arm of the present invention, radicals chosen, for example, from H, O, alkyl, aryl, and a heteroaryl each containing from 2 to 20 carbon atoms possibly being attached to these atoms.

This spacer arm (I) can be used, in general, for attaching a molecular unit [mo] to a solid support [Sup], for example so as to fabricate a biochip, or more advantageously a glycochip, where [mo] is generally a molecule which functionalizes said biochip.

According to the invention, preferably, in the spacer arm [1] as defined above:
X⁰ and X⁴ can each be chosen, independently of the other substituents, from C, O, N, S and Si; and/or
X¹; X²; and X³ can each be chosen, independently of the other substituents, from C, O, N, S and Si, and from an aryl and a heteroaryl each containing, for example, from 2 to 10 carbon atoms; and/or
Z¹ and Z² can each be chosen, independently of the other substituents, from C, N, C—R and Si—R, where R is an alkyl containing from 1 to 30 carbon atoms, preferably from 1 to 20 carbon atoms, preferably from 1 to 10 carbon atoms; and/or
R¹; R²; and R³ can each be chosen, independently of the other substituents, from H, an alkyl, an aryl and a heteroaryl each containing from 2 to 10 carbon atoms.

According to the invention, n, m and p can also be chosen, independently of one another, such that 1≦n, m and p≦30, preferably such that 1≦n, m and p≦20, and more preferably such that 1≦n, m and p≦10.

By way of example, according to the invention, in the spacer arm [1] as defined above, X⁰ and X⁴ are C; X¹, X², and X³ are C; Z¹ and Z² are C; and R¹, R², and R³ are H.

According to the invention, the protective group [Gp] can be any one of the groups which protect secondary amines known to those skilled in the art. It is preferably chosen such that it withstands the chemistry for synthesizing the spacer arm, for attaching it to the support and for attaching it to the molecular unit [mo]. It can be chosen, for example, from Ac, Bn (benzyl), a C₁ to C₄₀ aryl group (R), Troc, z, TCA, BOC, Fmoc, etc., so as to form, with the secondary amine of the spacer arm (I), one of the following chemical groups (>N— indicates the protected secondary amine):

>N-Ac: acetamide (>N—CO—Me);

>N-Bn: benzylamide;

>N—R: C₁ to C₄₀ arylamide;

>N-Troc: 2,2,2-trichloroethyl carbamate (>N—C(O)OCH₂CCl₃);

>N-z: benzyl carbamate (>N—C(O)OCH₂Ph);

>N-TCA: trichloroacetamidate (>N—CO—CCl₃);

>N—BOC: t-butyl carbamate (>N—C(O)OCMe₃);

>N-Fmoc: 9-fluorenylmethyl carbamate: 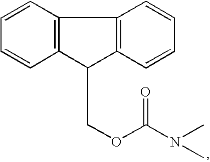
(Ph=phenyl and Me=methyl).

Preferably, according to the invention, the protective group is chosen from Ac, BOC or a C₁ to C₄₀ aryl group.

According to the invention, the molecule [Gp] participating in the functionality of the spacer arm can, for example, be a C₁ to C₄₀, for example C₁ to C₃₀, for example C₁ to C₂₀ or C₁ to C₁₀, alkyl or aryl. It can be any substituent, not necessarily protective, which can participate in the functionality of the spacer arm when it is used. It can, for example, be a hydrophobic group, making it possible to render the spacer arm more specific and/or more selective with respect to the molecule [mo] to be attached, and/or to its role during the use of the spacer arm, for example on a glycochip or a protein chip.

According to the invention, the solid support can, for example, be any support that can be silanized. It may, for example, be plates, beads or capillaries. It may, for example, be based on silica, on glass, or on other materials known to those skilled in the art, for example for producing biochip supports or surfaces. The silanization of the support can be carried out by any process known to those skilled in the art.

According to the invention, the molecular unit [mo] can be a natural or synthetic molecule. It can be any molecule which must be attached to a support, for example for analytical reasons. It may be a small molecule, for example having a molecular weight ranging from approximately 180 to 400 000 g.mol⁻¹. When it is a sugar, [mo] can, for example, have a molecular weight ranging from 180 to 10 000 g.mol⁻¹. When it is a protein or a peptide, [mo] can, for example, have a molecular weight ranging from 5500 to 400 000 g.mol⁻¹, generally ranging from 5500 to 220 000 g.mol⁻¹ (molecular weight of most proteins).

This molecular unit [mo] can, for example, be chosen from monosaccharides, oligosaccharides, polyoligosaccharides, glycoconjugates, peptides, proteins, enzymes, glycoproteins, lipids, fatty acids, glycolipids, glycolipoproteins, etc.

Among monosaccharides, mention may be made of glucose, glucosamine, azidoglucosamine, D-ribose, D-xylose, L-arabinose, D-glucose, D-galactose, D-mannose, 2-deoxy-D-ribose, L-fucose, N-acetyl-D-glucosamine, N-acetyl-D-galactosamine, N-acetyl-neuraminic acid, D-glucuronic acid, L-iduronic acid, D-sorbitol, D-mannitol, etc.

Among oligosaccharides, mention may be made of sucrose, lactose, heparan sulphate fragments, saccharide fragments of heparin, of chondroitin, of dermatan sulphates, Lewis antigens, etc.

Among polyoligosaccharides, mention may be made of saccharide portions of heparan sulphates, of heparin, of chondroitin, dermatan sulphates, etc.

Among glycoconjugates, mention may be made of heparan sulphates, heparin, chrondroitin, dermatan sulphates, etc.

Among peptides and proteins, mention may be made of chemokines, cytokines, insulin, fibrinogen, myosin, haemoglobin, etc.

Among enzymes, mention may be made of oxidoreductases, transferases, hydrolases, lyases, isomerases, ligases.

Among glycoproteins, mention may be made of immunoglobulin G, hyaluronic acid, etc.

Among lipids, mention may be made of hydrolysable lipids: fats (glycerol+3 fatty acids), waxes (fatty acid+fatty alcohols), sterol esters (sterol+fatty acids), phospholipids (phosphatidic acids (glycerol, 2 fatty acids+phosphate)), phosphalides (glycerol+2 fatty acids+phosphate), sphingolipids (sphingosine+fatty acid+phosphate+amino alcohol); non-hydrolysable lipids: alkanes, carotenoids, sterols (cholesterol), steroids (estradiol, testosterone), acids (fatty acids), eicosanoids, etc.

Among fatty acids, mention may be made of arachidonic acid, linoleic acid, linolenic acid, lauric acid, nervonic acid, palmitic acid, oleic acid, etc.

Among glycolipids, mention may be made of galactosylceramide, glucosylceramide, gangliosides, cerebrosides (fatty acid+sphingosine+1 sugar), gangliosides (fatty acid+sphingosine+numerous sugars, neuraminic acid), etc.

Among glycolipoproteins, mention may be made of MPB83 (trade mark), GLP19 (trade mark) and IRBP (trade mark).

The present invention also relates to a process for the covalent attachment of a molecular unit [mo] to a solid support by means of a spacer arm, advantageously that of the present invention.

The process can comprise the following steps: (i) reduction of the nitrile function of a compound of formula: 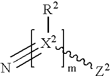
(ii) formation of an aldehyde function from an allyl function of a biological molecule of formula: 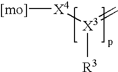
(iii) reductive amination, followed by protection of the secondary amine formed, between said reduced nitrile function and said aldehyde function, so as to obtain a biological molecule which has been activated so as to be attached to the support, said activated biological molecule being of formula: 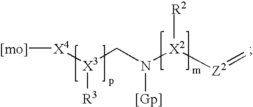
(iv) silanization of a solid support, and functionalization of the silanized solid support with a molecule of formula: 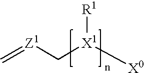
(v) metathesis reaction between the molecule functionalizing the support and the activated biological molecule so as to form a spacer arm according to the invention connecting the biological molecule and the support.

In this process, the substituents X⁰; X¹; X²; X³; X⁴; Z¹; Z²; R¹; R²; R³; and [mo] are as defined above.

According to the invention, the compound of formula 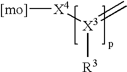
can, for example, be an allylated sugar, [mo] being said sugar. This allylated sugar can be obtained by any process known to those skilled in the art which does not impair the sugar. It may, for example, be the process described in document [5].

According to the invention, the secondary amine can also be protected with a protective group. Thus, the process of the invention can also comprise a step consisting of attachment of a protective group [Gp] to the secondary amine function. The protective group may be as defined above. The attachment thereof to the secondary amine can be carried out by any chemical process known to those skilled in the art, for example according to one of the processes described in document [7].

To carry out the various steps of this process of the invention, conventional organic chemistry processes known to those skilled in the art can be used. Thus, by way of example, for the step consisting of reduction of the nitrile, the process described in document [6] can be used. For the step consisting of formation of an aldehyde function from an allyl function of a biological molecule, the ozonolysis process described in document [5] can be used. For the step consisting of reductive amination followed by protection of the nitrogen, between the reduced nitrile and said aldehyde function, so as to obtain an activated biological molecule, the process described in document [7] can be used. For the step consisting of silanization of the solid support and functionalization thereof, the process described in document [8] can be used. For the metathesis reaction, the process described in document [10] can be used.

The spacer arm of the present invention can therefore be created from three parts which are linked, firstly, by reductive amination followed by protection of the nitrogen on the molecular unit side, and, secondly, in a Grubbs metathesis reaction. The Grubbs metathesis is, for example, described in document [11].

The nitrogen atom which is inserted into the carbon chain has several advantages: obtained during the attachment of two chain members, it is in the form of a secondary amine which can be protected in various ways so as to confer a specific reactivity on the spacer. This function, which can be advantageously modulated case by case with various protective groups or with a molecule which participates in the functionality of the spacer arm, makes it possible to vary and control the hydrophilicity or the hydrophobicity of the spacer arm and to control the steric hindrance thereof. It is also advantageously possible to modulate the electrophilic/nucleophilic or acidic/basic nature of this part of the spacer arm: the nature of the nitrogen atom-protecting groups is therefore preferably chosen with the aim of optimizing conditions for reactions, interactions, operations consisting of characterizations or analyses, whether before or after cleavage of the spacer so as to release the molecular unit. For example, with an acetyl group, because of its small size, a low steric hindrance is obtained, which allows optimization of the molecular recognition when the spacer arm is used, for example on a molecule chip. For example also, with a butyl group, a hydrophobic carbon-based substituent which renders this part of the spacer arm hydrophobic is obtained, which allows, for example, a recognition of hydrophilic proteins which is more specific and more selective with respect to the hydrophilic parts of the spacer arm ([mo]).

The present invention therefore provides a modulatable spacer arm (or “spacer”), the various structures of which influence the reactivity of the arm, i.e. the chemical and/or electrochemical and/or steric behaviour thereof.

The present invention can be realized simply and effectively and the spacer advantageously has the following three properties, in particular when it is used for the fabrication of glycochips:
first of all, the spacer successfully performs the function of an arm for distancing the glycol chain from the solid surface which supports this chain;
next, the spacer is a cleavable arm: it is possible to readily open the spacer, in a targeted manner, so as to isolate the sugar from the supported phase;
finally, the spacer, by virtue of its low degree of chemical functionality, remains inert under numerous conditions for reactions carried out during organic syntheses on glyco units, for example, and when glycochips are used.

Besides the abovementioned advantages, the inventors have noted the following during the various experiments carried out, of the present invention:
The spacer arm makes it possible to overcome the steric problems due to the presence of the solid support. It makes it possible to study, under good steric conditions, protein/sugar interactions on the glycochips obtained. It solves the problems of steric hindrance which were exhibited in the prior art when the protein approached the glyco ligands, and were harmful to the future potential interactions.
The length of the arm can be modulated: a judicious choice of functional homologues of different sizes, in particular through the choice of the starting reactants, makes it possible to prepare spacers of different sizes.
It is not only possible to choose the distance between the glyco chain and the solid support, but also to control the hydrophilic or hydrophobic nature of this part of the spacer by means of the protective group.
The simplicity of the chemical structure of the spacer confers on it properties of chemical non-reactivity during the numerous organic reactions when it is fabricated and when the glycochip is used.
The spacer, because of its lack of interactive chemical functions, has no influence on potential interactions with other molecules, when the system is used in the context of a glycochip or, more generally, of a small-molecule chip.

The spacer can be precisely and selectively cleaved at its C═C double bond, under reaction conditions which do not impair the biological molecule, for example oligosaccharide molecule. In fact, an ozonolysis (O₃), a Grubbs metathesis (Grubbs catalyst), or a dihydroxylation followed by a diol osmylation oxidative cleavage (OsO₄, NaIO₄), and other mild chemical reactions known to those skilled in the art, for example, can be conveniently used for the cleavage.
The fact that the spacer is readily cleavable, and that this cleavage does not modify the structure of the sugar, makes it possible to carry out structural and conformational analytical controls on the isolated oligosaccharide chain. It is also easy to calculate the amount of glyco probes attached to the solid support (“loading”) during the glyco synthesis.

One of the advantages of this spacer, in comparison with that described in document [1] (octenediol for example) lies in the adaptability of its length, of its functionality, of its reactivity and of the steric hindrance which can be generated as desired.

The inventors also note that the spacer of the present invention allows bonding with a very large range of saccharides, of oligosaccharides or of polysaccharides which are very commonly presynthesized and protected in the anomeric position, with respect to their reducing portion, with an allyl group. These glyco units can in fact be converted, in one step, so as to bind directly to the spacer. Thus, this spacer is advantageously compatible with numerous glyco molecules as have already been synthesized and described in the literature, for example in documents [7], [12] and [13].

The present invention can, for example, be used for the manufacture of a glycochip, for example of a chip capable of identifying by screening oligosaccharide sequences which recognize a specific protein, for example according to the technique described in document [1]. In this application, the present invention makes it possible to optimize the screening processes and therefore to have, more effectively and more rapidly, molecules for therapeutic or biotechnological purposes. One may expect this capacity to also exist in the other applications of the present invention.

The present invention can also be used on biochips where a spacer arm must form the link between the solid support and oligopeptide probes, oligonucleotide probes and/or oligosaccharide probes. In particular, the spacer arm of the present invention can be used on an oligopeptide chip such as that described in document [3], or on an oligosaccharide chip such as that described in document [4].

Other characteristics and advantages will also become apparent to those skilled in the art on reading the examples which follow, given by way of illustration.

### EXAMPLES

1) By way of example, the synthesis of a spacer having a length corresponding to a chain of fourteen carbons is disclosed below, using procedures chosen from those accessible to those skilled in the art. The references in bold characters refer to the reaction scheme below.

The compounds chosen are: a monosaccharide (1) of the glucose type (1) (N-acetylglucosamine (GlcNac): sugar allylated in the 1-position) constituting the molecular unit [mo]; 4-pentenenitrile (3) bearing the nitrile function to be reduced; and 7-octenyltrimethoxysilane (8) for the functionalization of the support. The solid support consists of silica-based Controlled Pore Glass (CPG) (trade mark) beads (6).

The reaction scheme below summarizes all the chemical reactions undertaken in these examples for the attachment of an oligosaccharide (1) to a support (6) by means of a spacer arm in accordance with the present invention. These chemical reactions are indicated by the letters A to F. An example of a reaction consisting of cleavage of the spacer arm is presented in Example G below.

On this reaction scheme, the groups “R” indicated on the sugar have intentionally not been differentiated in order to simplify the representation. These “R” groups represent substituents, which may be identical to or different from one another, at the various positions on the ring forming the sugar, and which may be the substituents generally encountered on sugars. In the specific example presented here, the sugar used being N-acetylglucosamine, those skilled in the art have no difficulty in identifying the substituents “R” at the various positions of compounds (1), (2), (5) and (10).

### Example A

### Activation of the Oligosaccharide (Reaction A)

An ozonolysis reaction is used in this example. The process used is described in document [5].

The sugar allylated in the anomeric position (1) (0.93 mmol) is dissolved in 5 ml of a mixture of dichloromethane and methanol (1/1): the medium is immersed in a cold bath at a temperature of −78° C. (acetone+dry ice). The ozone O₃ must then sparge in the solution: as soon as the blue colour (characteristic of an excess of ozone) appears, the ozone is replaced with argon (or nitrogen). Since the reaction is complete, the medium is rendered reducing by the addition of dimethyl sulphide Me₂S (4.65 mmol, 5 eq): dimethyl sulphoxide DMSO then forms. The medium slowly goes back up to ambient temperature overnight, and is then evaporated under vacuum: the organic residue is taken up with diethyl ether Et₂O, and washed with water. The organic phases are evaporated under vacuum, and then co-evaporated with toluene. The crude product is purified by silica gel column chromatography (eluent: petroleum ether/ethyl acetate: 8/2).

Thus, the aldehyde (2) is obtained with a yield of 75%.

### Example B

### Reduction of a Nitrile (Reaction B)

The chemical process used is described in document [6].

Lithium aluminium hydride LiAlH₄ (381 mg, 10.03 mmol, 1 eq) is introduced into freshly distilled diethyl ether (20 ml).

The 4-pentenenitrile (3) (814 mg, 1 ml, 10.03 mmol) is added slowly to the reaction medium, stirred under a nitrogen atmosphere, at a temperature of 0° C. (ice bath). The stirring must continue for approximately 20 minutes at ambient temperature.

Next, water (0.4 ml), then a solution of sodium hydroxide at 20% in water (0.3 ml) and, finally, another amount of water (1.4 ml) are added: these additions must be carried out with a great deal of care since the neutralization can be violent. When the solution of diethyl ether is separated, by settling out, from the inorganic white residue, the supernatant is extracted.

The white solid (residue) is washed twice with diethyl ether, and the organic phases are combined. A 3 M solution of hydrochloric acid HCl is added to this organic phase so as to obtain an acidic pH (pH<7): the 4-pentenenitrile which has not reacted remains in the ethereal phase, whereas the amine goes into the aqueous phase.

After extraction, the aqueous phase is therefore conserved and a 3 M solution of sodium hydroxide NaOH is added thereto so that the pH changes to a basic pH (pH>7): the aminated product will then go into the ethereal phase during this new extraction. The ethereal phase thus extracted is dried over magnesium sulphate (MgSO₄), and then evaporated under vacuum (rotary evaporator).

The crude amine (4) is then purified by fractionated distillation (bulb oven, T≈96° C.±9° C.).

¹H NMR analysis (Brücker AM 250):

5.82 (ddt, ³Jₜᵣₐₙₛ=18 Hz, ³J_{cis}=13 Hz, ³J(H^{I})=6.5 Hz, 1H, CH═), 5.00 (m, 2H, CH₂═), 2.70 (t, ³J_{II})=6.5 Hz, 2H, CH_{III}), 2.10 (ttd, ³J(H_{II})=6.5 Hz, ³J(HC)=6.5 Hz, ³J(H₂C—)=1.5 Hz, 2H, CH_{I}), 1.70 (s, 2H, NH₂), 1.56 (quint., ³J(H_{II})=³J(H_{III})=6.5 Hz, 2H, CH_{II}).

¹³C NMR analysis (Brücker AM 250):

138.6 (CH═), 114.6 (CH₂═), 42.0 (CH₂—N), 33.1 (CH₂), 31.4 (CH₂).

### Example C

### Reductive Amination (Reaction C)

The chemical process used is described in document [7].

The aldehyde (2) (20.87 mmol) is dissolved in dimethylformamide (1.2 ml) freshly distilled over calcium hydride (CaH₂): the medium is stirred and the amine (4) (31.30 mmol, 2 eq) is added. After about twenty minutes, sodium cyanoborohydride NaBH₃CN (83.47 mmol, 4 eq) is added to the mixture, which is left to stir at ambient temperature overnight.

If the reaction is not complete, it is possible to add NaBH₃CN (1 eq) again. Next, when the reaction is complete, pyridine (2.4 ml) and acetic anhydride Ac₂O (83.47 mmol, 2 eq/amine) are added to the mixture.

When the reaction is complete (approximately 1 hour after the addition), the crude compound is extracted with diethyl ether and with water. The combined organic phases are dried over magnesium sulphate (MgSO₄), and then filtered, evaporated under vacuum, and then co-evaporated with toluene.

The compound (5) is then purified by silica gel chromatography (gradient of eluent cyclohexane/ethyl acetate from 7/3 to 5/5).

### Example D

### Functionalization of the Solid Support (Reactions D and E)

The chemical process used is described in document [7].

The Controlled Pore Glass beads (6) (CPG, 500 Å, 2 g) are stirred very gently for 2 hours at ambient temperature in a solution of sodium hydroxide NaOH (700 mg) in deionized water DIW (6 ml) and 99% ethanol EtOH (8 ml). The beads are then centrifuged, the supernatant is extracted, and the beads are washed abundantly with DIW so as to attain a neutral pH.

The beads are then dried under vacuum (rotary evaporator), and remain at ambient temperature for 1 hour in a solution of hydrochloric acid (0.2 N HCl) before being washed with water, centrifuged, dried, and then placed in an incubator for 15 minutes at 80° C. They are then washed with ethanol, and then with toluene (centrifuge).

They are then dried before participating in the subsequent silanization step, for which the reaction mixture will have been prepared just before use.

The CPG beads (7) are introduced into a mixture of toluene (45 ml), triethylamine Et₃N (1.35 ml) and 7-octenyltrimethoxysilane (8) (C₁₁H₂₄O₃Si, M 232.39, 100 μl): the reaction medium is placed at 80° C. for 16 hours (incubator).

The beads are extracted from the mixture by centrifugation, and are rinsed with ethanol several times and then dried (rotary evaporator). They are then subjected to a temperature of 110° C. for 3 hours in order to perform the crosslinking step (incubator).

The silanization and crosslinking steps having thus been carried out, it is necessary to neutralize the residual acidity and hydrophilicity of the surface silanols which have not reacted during the silanization step (“end-capping”). A solution of trimethylsilyl chloride TMSCl (109 mg, 130 μl) and triethylamine Et₃N (506 mg, 700 μl) in dichloromethane DCM (10 ml) is added to the silanized CPG beads, and the mixture is left to stir gently for 2 hours at 25° C.

The beads are then rinsed abundantly with dichloromethane (centrifuge) and then with acetonitrile (centrifuge). They are then dried under vacuum (rotary evaporator) and placed in an incubator (80° C.) so as to complete the drying.

The silanized beads (9) are thus obtained.

### Example F

### Metathesis Reaction (Reaction F)

The chemical process used is described in document [9].

The silanized CPG beads (9) (2 g, 30 μmol/g) are stirred in dichloromethane (20 ml), under a nitrogen atmosphere. The sugar-spacer system (5) (300 μmol, >5 eq) is then added, to the medium, with a Grubbs catalyst (6 μmol, 5 mg, 0.1 eq). The reaction medium is then brought to reflux, i.e. a temperature of 44° C.

After 6 hours, another portion of Grubbs catalyst (6 μmol, 5 mg, 0.1 eq) is added. The mixture is maintained at 44° C. for a further 6 hours, and is then returned to ambient temperature.

The beads are filtered off, and washed abundantly with dichloromethane and with ethanol (centrifuge). The beads are then evaporated under vacuum so as to be dried.

The glyco beads (10) are thus obtained.

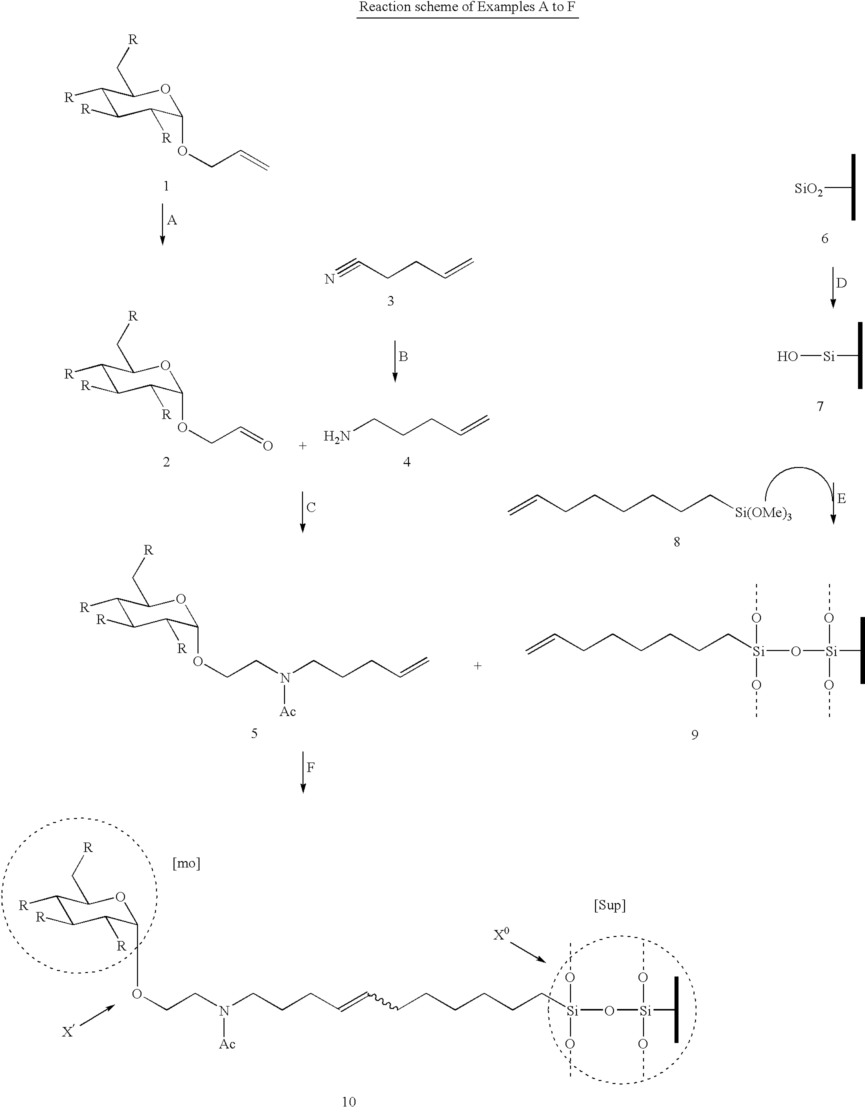

### Example G

### Cleavage of the Probe (Reaction G)

The chemical process used is described in document [10].

When the system (10) (solid support-spacer of the present invention-oligosaccharide chain) has been obtained, it is possible to cleave the spacer, without denaturing the glyco chain.

The experimental protocol is described in document [5]. The chemical equation is as follows:

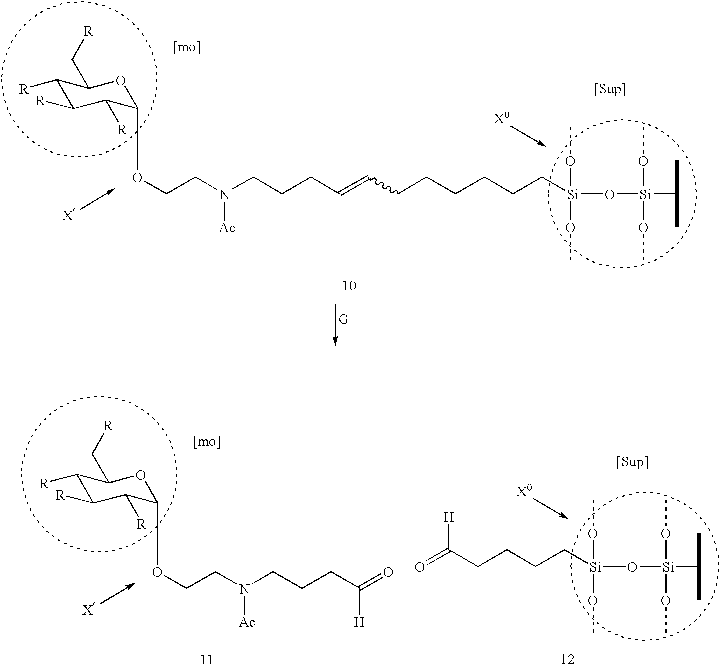

The glyco CPG beads (10) are stirred slowly in a 1/1 dichloromethane/methanol mixture. The medium is brought to a temperature of −78° C. (acetone+liquid nitrogen).

Ozone O₃ is then bubbled into the reaction medium until a blue colour appears.

Next, argon is bubbled into the mixture for a few minutes, before neutralizing the medium with dimethyl sulphide, and the reaction medium is then left to come back up to ambient temperature overnight.

The beads are taken up with diethyl ether, filtered, and rinsed several times with diethyl ether and with water.

The beads (12) are then put aside and the supernatant is extracted (diethyl ether/water) and the organic phase is dried over magnesium sulphate MgSO₄, evaporated under vacuum and co-evaporated with toluene.

The product (11) is then obtained.

2) Other molecules according to the invention have been produced using the procedures disclosed above (Examples A to F). These molecules are described in detail in the following examples.

### Example H

In this example, [mo] is an RGD (Arg-Gly-Asp) peptide attached to the spacer arm of the invention via its C-terminal end. The support is the same as in the procedure disclosed above. Thus, in this example, the molecule (1) of the reaction scheme of Examples A to F is replaced with RGD.

Beads to which the RGD peptide is attached are thus obtained. They have the formula: 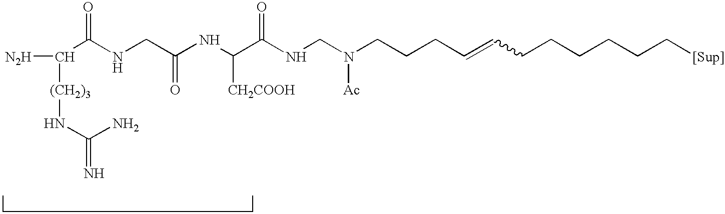
[mo]: RGD peptide attached via the C-terminal (Arg-Gly-Asp)

### Example I

This example uses the same [mo] as in Example H, but attached to the spacer arm of the invention via its N-terminal end. Furthermore, in 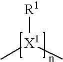
of the spacer arm of the invention, X¹ is C and n=20. The term “C” is of course intended to mean a hydrogenated carbon.

The beads obtained have the formula: 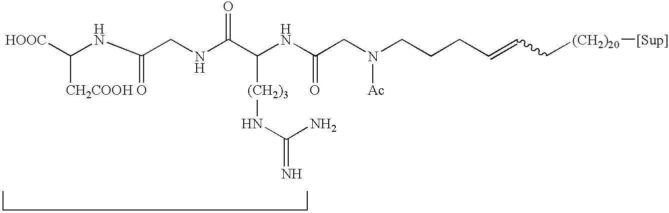
[mo] is an RGD peptide attached via the N-terminal end

### Example J

In this example, [mo] is a “sialyl-Lewis a”.

The support is the same as in the procedure disclosed above. The protective group is Boc. Its attachment chemistry is known to those skilled in the art.

Beads to which sialyl-Lewis a is attached are thus obtained. They have the formula: 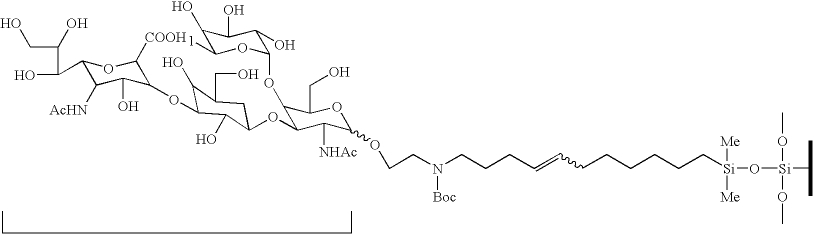
[mo] is a sialyl-Lewis a, with “1”=H or CH₃

### Example K

In this example, [mo] is a sulphated compound.

The support is the same as in the procedure disclosed above.

Beads to which the sulphated compound is attached are thus obtained. They have the formula: 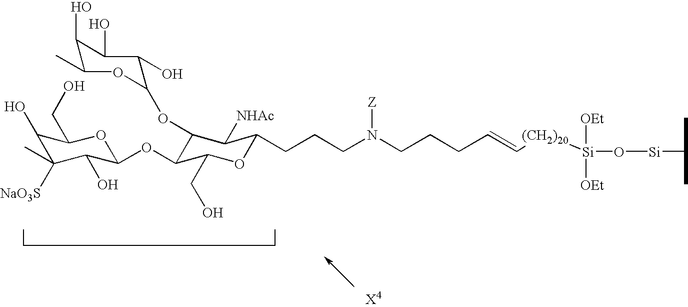
[mo] is a sulphated compound,
Z being a protective group (for example Gp defined in the “disclosure of the invention” section)

In another protocol, the carbon X⁴ was replaced with a sulphur atom. The corresponding beads were obtained.

### Example L

In this example, [mo] is a protected sugar. The support is the same as in the procedure disclosed above.

Beads to which the protected sugar is attached are thus obtained. They have the formula: 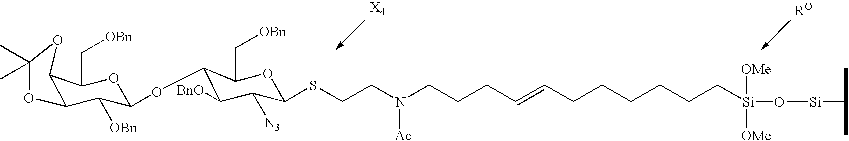
protected sugar

### Example M

In this example, [mo] is a sialic acid. The support is the same as in the procedure disclosed above.

Beads to which sialic acid is attached are thus obtained. They have the formula:

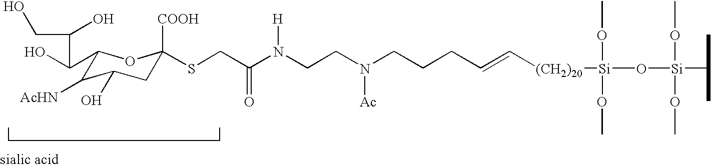

### BIBLIOGRAPHICAL REFERENCES

[1] WO-A-03/008927: Dukler, N. Dotan, A. Shtavi, A. Gargir.
[2] H. M. I. Osborn, T. H. Khan, Tetrahedron, 1999, 55, 1807-1850.
[3] D. A. Stetsenko, M. J. Gai, Bioconjugate Chemistry, 2001, 12, 576-586.
[4] U.S. Pat. No. 6,579,725: P. H. Seeberger, R. B. Andrade.
[5] R. Roy, C. A. Laferrière, Canadian Journal of Chemistry, 1990, 68, 2045-2054.
[6] L. H. Amundsen, L. S, Nelson, Journal of the American Chemical Society, 1951, 73, 242-244.
[7] J. F. Tolborg, K. J. Jensen, Chemical Communication, 2000, 147-148.
[8] F. Vinet, A. Hoang, EN 00 16940.
[9] K. Biswas, D. M. Coltart, S. J. Danishefsky, Tetrahedron Letters, 2002, 43, 6107-6110.
[10] C. Sylvain, A. Wagner, C. Miokowski, Tetrahedron Letters, 1997, 38, 1043-1044.
[11] Q. J. Plante, E. R. Palmacci, P. H. Seeberger, Science, 2001, 291, 1523-1527.
[12]P. H. Seeberger, Chem. Com., 2003, 1115-1121.
[13] D. M. Ratner, E. R. Swanson, P. H. Seeberger, Org. Lett. 2003, 4717-4720.

1-26. (canceled)

27. Molecular spacer arm of formula (I) below: 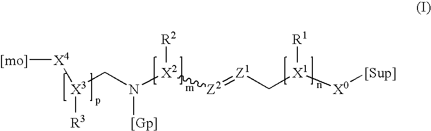
wherein X⁰ and X⁴ are substituents which can be modulated so as to allow bonding of [mo] and [Sup] via said spacer arm, X⁰ and X⁴ being different from H and each being chosen, independently of the other substituents of the spacer arm, from C, O, N, S, Se, P, As and Si; and
wherein the substituents X¹, X², X³, Z¹, Z², R¹, R², and R³ are such that:
X¹, X², and X³ are each chosen, independently of the other substituents, from C, O, N, S, Se, P, As and Si, and from an aryl and a heteroaryl, each containing from 2 to 20 carbon atoms;
Z¹ and Z² are each chosen, independently of the other substituents, from C—R, Si—R, C, N, P and As, where R is an alkyl containing from 1 to 40 carbon atoms;
R¹, R², and R³ are each chosen, independently of the other substituents, from H, an alkyl, an aryl and a heteroaryl each containing from 2 to 20 carbon atoms;
[Gp] represents a group which protects the secondary amine —N— or a molecule which participates in the functionality of the spacer arm;
wherein n, m and p are integers, each greater than or equal to 1 and chosen independently of one another;
wherein [Sup] represents H or a silanized solid support; and
wherein [mo] represents H or a molecular unit.

28. Molecular spacer arm according to claim 27 wherein 1≦n, m and p≦40.

29. Molecular spacer arm according to claim 27, wherein
X⁰ and X⁴ are chosen, independently of the other substituents, from C, O, N, S and Si; and/or
X¹, X², and X³ are chosen, independently of the other substituents, from C, O, N, S and Si, and from an aryl and a heteroaryl each containing from 2 to 10 carbon atoms; and/or
Z¹ and Z² are chosen, independently of the other substituents, from C, N, C—R and Si—R, where R is an alkyl containing from 1 to 30 carbon atoms; and/or
R¹, R², and R³ are chosen, independently of the other substituents, from H, an alkyl, an aryl and a heteroaryl each containing from 2 to 10 carbon atoms.

30. Molecular spacer arm according to claim 27, wherein the protective group [Gp] is chosen from Ac, benzyl, a C₁ to C₄₀ aryl group, Troc, z, TCA, BOC and Fmoc.

31. Molecular spacer arm according to claim 27, wherein the solid support [Sup], when it is present, is chosen from a plate, a bead or a capillary.

32. Molecular spacer arm according to claim 27, wherein [Sup] is silica-based or glass-based.

33. Molecular spacer arm according to claim 27, wherein [mo], when it is present, is a molecule having a molecular weight ranging from 180 to 400 000 g.mol⁻¹.

34. Molecular spacer arm according to claim 27, wherein [mo], when it is present, is chosen from monosaccharides, oligosaccharides, polyoligosaccharides, glycoconjugates, peptides, proteins, enzymes, glycoproteins, lipids, fatty acids, glycolipids and glycolipoproteins.

35. Molecular spacer arm according to claim 27, wherein [mo], when it is present, is a sugar.

36. A process for attaching a molecular unit [mo] to a silanized solid support [Sup] comprising covalently attaching the molecular unit to the silanized solid support through a molecular spacer arm according to formula (I): 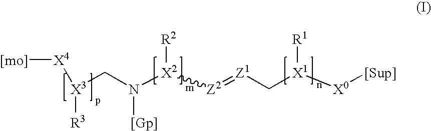
wherein X⁰ and X⁴ are substituents which can be modulated so as to allow bonding of [mo] and [Sup] via said spacer arm, X⁰ and X⁴ being different from H and each being chosen, independently of the other substituents of the spacer arm, from C, O, N, S, Se, P, As and Si; and
wherein the substituents X¹, X², X³, Z¹, Z², R¹, R², and R³ are such that:
X¹, X², and X³ are each chosen, independently of the other substituents, from C, O, N, S, Se, P, As and Si, and from an aryl and a heteroaryl, each containing from 2 to 20 carbon atoms;
Z¹ and Z² are each chosen, independently of the other substituents, from C—R, Si—R, C, N, P and As, where R is an alkyl containing from 1 to 40 carbon atoms;
R¹, R², and R³ are each chosen, independently of the other substituents, from H, an alkyl, an aryl and a heteroaryl each containing from 2 to 20 carbon atoms;
[Gp] represents a group which protects the secondary amine —N— or a molecule which participates in the functionality of the spacer arm; and
wherein n, m and p are integers, each greater than or equal to 1 and chosen independently of one another.

37. A process according to claim 36, wherein [mo] is a molecule having a molecular weight ranging from 180 to 400 000 g.mol⁻¹.

38. A process according to claim 36, wherein [mo] is chosen from monosaccharides, oligosaccharides, polyoligosaccharides, glycoconjugates, and natural or synthetic small molecules.

39. A process according to claim 36, wherein [Sup] is chosen from a plate, beads or a capillary.

40. A process according to claim 39, wherein [Sup] is silica-based or glass-based.

41. A process for producing a biochip comprising attaching a molecular unit [mo] to a silanized solid support [Sup], by a process comprising covalently attaching the molecular unit to the silanized solid support through a molecular spacer arm according to formula (I): 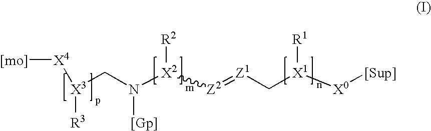
wherein X⁰ and X⁴ are substituents which can be modulated so as to allow bonding of [mo] and [Sup] via said spacer arm, X⁰ and X⁴ being different from H and each being chosen, independently of the other substituents of the spacer arm, from C, O, N, S, Se, P, As and Si; and
wherein the substituents X¹, X², X³, Z¹, Z², R¹, R², and R³ are such that:
X¹, X², and X³ are each chosen, independently of the other substituents, from C, O, N, S, Se, P, As and Si, and from an aryl and a heteroaryl, each containing from 2 to 20 carbon atoms;
Z¹ and Z² are each chosen, independently of the other substituents, from C—R, Si—R, C, N, P and As, where R is an alkyl containing from 1 to 40 carbon atoms;
R¹, R², and R³ are each chosen, independently of the other substituents, from H, an alkyl, an aryl and a heteroaryl each containing from 2 to 20 carbon atoms;
[Gp] represents a group which protects the secondary amine —N— or a molecule which participates in the functionality of the spacer arm; and
wherein n, m and p are integers, each greater than or equal to 1 and chosen independently of one another.

42. A process for producing a glycochip comprising attaching a molecular unit [mo] to a silanized solid support [Sup] by a process comprising covalently attaching the molecular unit to the silanized solid support through a molecular spacer arm according to formula (I): 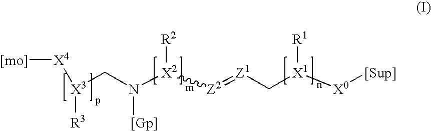
wherein X⁰ and X⁴ are substituents which can be modulated so as to allow bonding of [mo] and [Sup] via said spacer arm, X⁰ and X⁴ being different from H and each being chosen, independently of the other substituents of the spacer arm, from C, O, N, S, Se, P, As and Si; and
wherein the substituents X¹, X², X³, Z¹, Z², R¹, R², and R³ are such that:
X¹, X², and X³ are each chosen, independently of the other substituents, from C, O, N, S, Se, P, As and Si, and from an aryl and a heteroaryl, each containing from 2 to 20 carbon atoms;
Z¹ and Z² are each chosen, independently of the other substituents, from C—R, Si—R, C, N, P and As, where R is an alkyl containing from 1 to 40 carbon atoms;
R¹, R², and R³ are each chosen, independently of the other substituents, from H, an alkyl, an aryl and a heteroaryl each containing from 2 to 20 carbon atoms;
[Gp] represents a group which protects the secondary amine —N— or a molecule which participates in the functionality of the spacer arm; and
wherein n, m and p are integers, each greater than or equal to 1 and chosen independently of one another.

43. Process for the covalent attachment of a molecular unit [mo] to a support by means of a spacer arm, said process comprising the following steps:
(i) reduction of the nitrile function of a compound of formula: 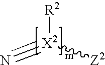
(ii) formation of an aldehyde function from an allyl function of a biological molecule of formula: 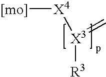
(iii) reductive amination, followed by protection of the secondary amine formed, between said reduced nitrile function and said aldehyde function, so as to obtain a biological molecule which has been activated so as to be attached to the support, said activated biological molecule being of formula: 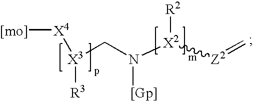
(iv) silanization of a solid support, and functionalization of the silanized solid support with a molecule of formula: 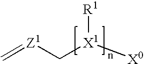
(v) metathesis reaction between the molecule functionalizing the support and the activated biological molecule so as to form a spacer arm connecting the biological molecule and the support;
wherein X⁰ and X⁴ are substituents which can be modulated so as to allow bonding of [mo] and the support via said spacer arm, X⁰ and X⁴ being different from H and each being chosen, independently of the other substituents of the spacer arm, from C, O, N, S, Se, P, As and Si; and
wherein the substituents X¹, X², X³, Z¹, Z², R¹, R², and R³ are such that:
X¹, X², and X³ are each chosen, independently of the other substituents, from C, O, N, S, Se, P, As and Si, and from an aryl and a heteroaryl, each containing from 2 to 20 carbon atoms;
Z¹ and Z² are each chosen, independently of the other substituents, from C—R, Si—R, C, N, P and As, where R is an alkyl containing from 1 to 40 carbon atoms;
R¹, R², and R³ are each chosen, independently of the other substituents, from H, an alkyl, an aryl and a heteroaryl each containing from 2 to 20 carbon atoms;
[Gp] represents a group which protects the secondary amine —N— or a molecule which participates in the functionality of the spacer arm; and
wherein n, m and p are integers, each greater than or equal to 1 and chosen independently of one another.

44. Process according to claim 43, in which the compound of formula 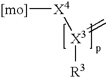
is an allylated sugar, [mo] being said sugar.

45. Process according to claim 43, in which [Sup] is chosen from a plate, a bead or a capillary.

46. Process according to claim 43, in which [Sup] is silica-based or glass-based.

47. Process according to claim 43, in which [mo] is a molecule having a molecular weight ranging from 180 to 400 000 g.mol⁻¹.

48. Process according to claim 43, in which [Mo] is chosen from monosaccharides, oligosaccharides, polyoligosaccharides, glycoconjugates, peptides, proteins, enzymes, glycoproteins, lipids, fatty acids, glycolipids and glycolipoproteins.

49. Process according to claim 43, in which [mo] is a sugar.

50. Process according to claim 43, further comprising a step consisting of attachment of a protective group [Gp] to the secondary amine function.

51. Process according to claim 50, wherein [Gp] is chosen from Ac, benzyl, a C₁ to C₄₀ aryl group, Troc, z, TCA, BOC and Fmoc.

52. A process for producing a biochip comprising covalently attaching a molecular unit [mo] to a support by means of a spacer arm by the following steps:
(i) reduction of the nitrile function of a compound of formula: 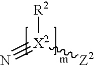
(ii) formation of an aldehyde function from an allyl function of a biological molecule of formula: 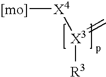
(iii) reductive amination, followed by protection of the secondary amine formed, between said reduced nitrile function and said aldehyde function, so as to obtain a biological molecule which has been activated so as to be attached to the support, said activated biological molecule being of formula: 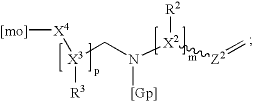
(iv) silanization of a solid support, and functionalization of the silanized solid support with a molecule of formula: 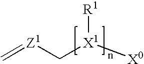
(v) metathesis reaction between the molecule functionalizing the support and the activated biological molecule so as to form a spacer arm connecting the biological molecule and the support;
wherein X⁰ and X⁴ are substituents which can be modulated so as to allow bonding of [mo] and the support via said spacer arm, X⁰ and X⁴ being different from H and each being chosen, independently of the other substituents of the spacer arm, from C, O, N, S, Se, P, As and Si; and
wherein the substituents X¹, X², X³, Z¹, Z², R¹, R², and R³ are such that:
X¹, X², and X³ are each chosen, independently of the other substituents, from C, O, N, S, Se, P, As and Si, and from an aryl and a heteroaryl, each containing from 2 to 20 carbon atoms;
Z¹ and Z² are each chosen, independently of the other substituents, from C—R, Si—R, C, N, P and As, where R⁴⁶ is an alkyl containing from 1 to 40 carbon atoms;
R¹, R², and R³ are each chosen, independently of the other substituents, from H, an alkyl, an aryl and a heteroaryl each containing from 2 to 20 carbon atoms;
[Gp] represents a group which protects the secondary amine —N— or a molecule which participates in the functionality of the spacer arm; and
wherein n, m and p are integers, each greater than or equal to 1 and chosen independently of one another.

53. A process for producing a glycochip comprising covalently attaching a molecular unit [mo] to a support by means of a spacer arm the following steps:
(i) reduction of the nitrile function of a compound of formula: 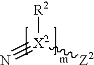
(ii) formation of an aldehyde function from an allyl function of a biological molecule of formula: 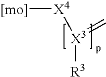
(iii) reductive amination, followed by protection of the secondary amine formed, between said reduced nitrile function and said aldehyde function, so as to obtain a biological molecule which has been activated so as to be attached to the support, said activated biological molecule being of formula: 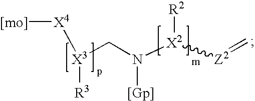
(iv) silanization of a solid support, and functionalization of the silanized solid support with a molecule of formula: 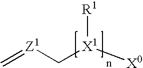
(v) metathesis reaction between the molecule functionalizing the support and the activated biological molecule so as to form a spacer arm connecting the biological molecule and the support;
wherein X⁰ and X⁴ are substituents which can be modulated so as to allow bonding of [mo] and the support via said spacer arm, X⁰ and X⁴ being different from H and each being chosen, independently of the other substituents of the spacer arm, from C, O, N, S, Se, P, As and Si; and
wherein the substituents X¹, X², X³, Z¹, Z², R¹, R², and R³ are such that:
X¹, X², and X are each chosen, independently of the other substituents, from C, O, N, S, Se, P, As and Si, and from an aryl and a heteroaryl, each containing from 2 to 20 carbon atoms;
Z¹ and Z² are each chosen, independently of the other substituents, from C—R, Si—R, C, N, P and As, where R is an alkyl containing from 1 to 40 carbon atoms;
R¹, R², and R³ are each chosen, independently of the other substituents, from H, an alkyl, an aryl and a heteroaryl each containing from 2 to 20 carbon atoms;
[Gp] represents a group which protects the secondary amine —N— or a molecule which participates in the functionality of the spacer arm; and
wherein n, m and p are integers, each greater than or equal to 1 and chosen independently of one another.
